(19) United States
(12) Patent Application Publication
(10) Pub. No.: US 2018/0185302 A1
(43) Pub. Date: Jul. 5, 2018

(54) METHODS FOR DOWNREGULATING APOBEC3B

(71) Applicant: Regents of the University of Minnesota, Minneapolis, MN (US)

(72) Inventors: Reuben S. HARRIS, St. Paul, MN (US); Brandon LEONARD, San Francisco, CA (US)

(21) Appl. No.: 15/738,651

(22) PCT Filed: Jun. 19, 2016
(86) PCT No.: PCT/US2016/040060

§ 371 Date: Dec. 21, 2017

Related U.S. Application Data
(60) Provisional application No. 62/187,643, filed on Jul. 1, 2015.
(60) Provisional application No. 62/186,462, filed on Jun. 30, 2015.

(51) Int. Cl. A61K 31/133; A61K 31/4045; A61K 31/407; A61K 31/506; A61K 31/517; A61K 31/5377; A61K 31/4743; A61K 31/437; A61K 31/473; A61K 31/381; A61K 31/7034; A61K 38/08; A61K 45/06; A61P 35/02; G01N 33/574; C12Q 1/6886

(52) U.S. Cl. CPC A61K 31/133; G01N 2333/978; A61K 31/407; A61K 31/506; A61K 31/517; A61K 31/5377; A61K 31/4743; A61K 31/437; A61K 31/473; A61K 31/381; A61K 31/7034; A61K 38/08; A61K 45/06; A61P 35/02; G01N 33/57484; C12Q 1/6886; C12Q 2600/106; C12Q 2600/156; C12Q 2600/158; A61K 31/4045

(57) ABSTRACT

In one aspect, a method of treating a subject having or at risk of having a tumor generally includes administering to the subject an amount of a PKC-NFkB axis inhibitor effective to ameliorate at least one symptom or clinical PMA sign of the tumor. In another aspect, a method of treating a subject having a tumor generally includes confirming that APOBEC3B is present in cells of the tumor and administering to the subject an amount of a PKC-NFkB axis inhibitor effective to decrease APOBEC3B in the cells of the tumor.

### CROSS-REFERENCE TO RELATED APPLICATION

This application claims priority to U.S. Provisional Patent Application No. 62/186,462, filed Jun. 30, 2015, and U.S. Provisional Patent Application No. 62/187,643, filed Jul. 1, 2015, each of which is incorporated herein by reference.

### SEQUENCE LISTING

This application contains a Sequence Listing electronically submitted via EFS-Web to the United States Patent and Trademark Office as an ASCII text file entitled “2016-06-28-SequenceListing_ST25.txt” having a size of 2 kilobytes and created on Jun. 28, 2016. The information contained in the Sequence Listing is incorporated by reference herein.

### SUMMARY

This disclosure describes a method of treating a subject having or at risk of having a tumor. Generally, the method includes administering to the subject an amount of a PKC-NFκB axis inhibitor effective to ameliorate at least one symptom or clinical sign of the tumor.

In some embodiments, the PKC-NFκB axis inhibitor can include a PKC inhibitor. In such embodiments, the PKC inhibitor can include Gö6983, Gö6976, MT477, RO 32-0432, myr-FARKGALRQ, chelerythrine, RO 31-7549, safingol, Compound 3, Compound 8, aprinocarsen, balmoralmycin (I), bisindolylmaleimides, or sotrastaurin.

In some embodiments, the PKC-NFκB axis inhibitor can include an NFκB inhibitor. In such embodiments, the NFκB inhibitor can include TPCA-1.

In some embodiments, the PKC-NFκB axis inhibitor can include a proteasome inhibitor.

In such embodiments, the proteasome inhibitor can include BAY 11-7082, MG132, bortezomib, salinosporamide A, or carfilzomib.

In some embodiments, the PKC-NFκB axis inhibitor comprises a NIK inhibitor.

In some embodiments, the tumor can be a tumor resulting from acute lymphoblastic leukemia (ALL), bladder cancer, breast cancer, cervical cancer, chondrosarcoma, chronic lymphocytic leukemia (CLL), esophageal cancer, head and neck cancer, kidney cancer, lung cancer, B cell lymphoma, melanoma, myeloma, osteosarcoma, ovarian cancer, pancreatic cancer, stomach cancer, thyroid cancer, uterine cancer, or uveal cancer.

In another aspect, this disclosure describes a method of treating a subject having a tumor. Generally, the method includes confirming that APOBEC3B is present in cells of the tumor and administering to the subject an amount of a PKC-NFκB axis inhibitor effective to decrease APOBEC3B in the cells of the tumor.

In some embodiments, the presence of APOBEC3B in the cells of the tumor is assayed by RT-qPCR, detecting an APOBEC3B mutation signature through DNA sequencing, or detecting the protein itself using an APOBEC3B-specific antibody.

In some embodiments, the PKC-NFκB axis inhibitor is administered to the subject after the subject receives another anti-tumor therapy.

In some embodiments, the PKC-NFκB axis inhibitor is administered to the subject before the subject receives another anti-tumor therapy.

In some embodiments, the PKC-NFκB axis inhibitor is administered to the subject concurrent with the subject receiving another anti-tumor therapy.

In some embodiments, the tumor therapy includes chemotherapy, targeted therapy, immunotherapy, radiotherapy, or palliative care.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. APOBEC3B upregulation by PMA. (A) A histogram showing the specific upregulation of APOBEC3B mRNA by PMA. MCF10A cells were treated with PMA (25 ng/ml) or vehicle control for six hours, and mRNA levels were measured by RT-qPCR (mean and SD are shown for triplicate RT-qPCR reactions normalized to TBP). The same data points are shown in the context of a larger PMA dose response experiment in FIG. 6. (B) A histogram demonstrating the dose responsiveness of APOBEC3B upregulation by PMA. Normalization and quantification were calculated as in FIG. 1A. The middle images show immunoblots for corresponding APOBEC3B and tubulin proteins levels, and the lower image shows DNA cytosine deaminase activity for the corresponding whole cell extracts (S, substrate; P, product; percent deamination quantified below each lane). (C) A histogram depicting the rapid kinetics of APOBEC3B upregulation following PMA treatment. MCF10A cells were treated with a single concentration of PMA (25 ng/ml), and mRNA, protein, and activity levels are reported as in FIG. 1B. (D) New protein synthesis is dispensable for APOBEC3B mRNA upregulation by PMA. Representative dose response experiment for MCF10A cells treated with the indicated concentrations of PMA following a 30-minute pretreatment with 10 μg/mL cyclohexamide. mRNA, protein, and activity levels are reported as in FIG. 1B.

FIG. 2. APOBEC3B upregulation by PMA is dependent on PKC. (A)-(F) Histograms reporting the impact of the indicated small molecules on PMA-induced APOBEC3B upregulation. APOBEC3B induction was inhibited by Gö6983 (pan-PKC inhibitor), BIM-1 (classical and novel PKC inhibitor), Gö6976 (classical PKC selective inhibitor), and AEB071 (preclinical PKC inhibitor), but not by LY294002 (PI3K inhibitor) or U0126 (MEK inhibitor). MCF10A cells were treated with PMA following a 30-minute pretreatment with the indicated concentrations of each inhibitor. mRNA expression is reported as the mean of three independent RT-qPCR reactions normalized to TBP (error bars report SD from triplicate assays). (G) Histogram depicting PKC isoforms expressed in MCF10A cells treated with PMA or vehicle control. mRNA expression was determined by RNA-seq and is reported as fragments per kilobase of exon per million fragments mapped (FKPM) and normalized to TBP. (H) Histogram showing that PKCα knockdown inhibits APOBEC3B induction by PMA. MCF10A cells were treated with PMA following PKCα knockdown using three independent PKCα specific shRNA encoding lentiviruses and a control. mRNA levels for both PKCα (blue) and APOBEC3B (red) are reported. (I) Immunoblots confirming PKCα knockdowns and proportional reductions in APOBEC3B protein levels.

FIG. 3. Non-canonical NFκB signaling is responsible for APOBEC3B upregulation by PMA. (A-B) Histograms depicting the dose responsive inhibition of PMA-induced APOBEC3B upregulation by BAY 11-7082 (ubiquitination inhibitor) and MG132 (proteasome inhibitor). MCF10A cells were treated with PMA following a 30-minute pretreatment with the indicated concentrations of each inhibitor. APOBEC3B mRNA expression is reported as the mean of three independent RT-qPCR reactions normalized to TBP (error bars report SD from triplicate assays). (C) Histogram depicting NFκB subunit mRNA levels in MCF10A cells treated with PMA or vehicle control. Expression was determined by RNA-seq and is reported as FKPM and normalized to TBP. (D) Plot depicting inhibition of PMA-induced APOBEC3B expression by the IκB kinase (IKK) inhibitor, TPCA-1, near the IC₅₀ for IKKα, not IKKβ. MCF10A cells were treated with PMA following treatment with varying concentrations of TPCA-1. TNFα (light) and APOBEC3B (dark) mRNA levels are reported as the mean of three independent RT-qPCR reactions normalized to TBP (error bars report SD from triplicate assays). The dotted lines denote previously reported in vitro IC₅₀ values for IKKα and IKKβ inhibition by TPCA-1 (Podolin et al., 2005, J Pharmacol Exp Ther. 312:373-381). (E) Histogram showing the kinetics of NFKBIA upregulation PMA. MCF10A cells were treated with PMA for the indicated times and mRNA values were quantified as in FIG. 3A. (F) The APOBEC3B and NFKBIA promoter regions contain several putative NFκB binding sites (TSS, transcriptional start site). (G) RELB and p105/p52 are specifically and robustly recruited to the APOBEC3B promoter region by PMA. ChIP was performed after a treatment with PMA or vehicle control for two hours. qPCR results are reported as percent of the total chromatin input.

FIG. 4. The PKC-NFκB pathway drives endogenous APOBEC3B expression in cancer cells. (A) APOBEC3B mRNA levels in representative breast, ovarian, and head/neck cancer cell lines. mRNA expression is reported as the mean of three independent RT-qPCR reactions normalized to TBP (error bars report SD from triplicate assays). (B) Representative PKC inhibitor treated cancer cell line experiment. Each line was treated with AEB071 (10 μM) or vehicle control for 48 hours prior to analysis. The histogram reports APOBEC3B mRNA levels normalized to the vehicle treated control for each line. The middle images show immunoblots for corresponding APOBEC3B and tubulin protein levels, and the lower image shows DNA cytosine deaminase activity for the corresponding whole cell extracts (S, substrate; P, product; percent deamination quantified below each lane).

FIG. 5. Model for APOBEC3B upregulation by the PKC-NFκB pathway. PKCα activation by DAG or PMA leads to IKKα phosphorylation and proteasome-dependent cleavage of NFκB subunit p100 into the transcriptionally active p52 form. The non-canonical NFκB heterodimer containing p52 and RELB is then recruited to the APOBEC3B promoter to drive transcription. Red labels represent the small molecules and approaches used to interrogate this signal transduction pathway.

FIG. 6. APOBEC family member mRNA levels in MCF10A cells treated with the indicated PMA concentrations or DMSO as vehicle control for six hours. mRNA expression is reported as the mean of three independent RT-qPCR reactions normalized to TBP (error bars report SD from triplicate assays). The 25 ng/ml data are shown in FIG. 1A.

FIG. 7. Exemplary non-canonical NF-κB pathway inhibitors. Chemical Structures of: (A) RO 32-0432; (B) Gö6976; (C) Gö6983; (D) chelerythrine; (E) MT477; (F) RO 31-7549; (G) safigol; (H) Compound 3 (Lee et al., 2005, Bioorg. Med. Chem. Lett. 15:2271-2274), which is a modification of SEQ ID NO:4; (I) Compound 8 (Lee et al., 2005, Bioorg. Med. Chem. Lett. 15:2271-2274)), which is a modification of SEQ ID NO:5; (J) balmoralmycin; (K) bisindolylmaleimide-1; (L) myr-FARKGALRQ; (M) sotrastaurin (AEB071); (N) TPCA-1; (O) BAY 11-7082; (P) MG132; (Q) bortezomib; (R) salinosporamide A; (S) cafilzomib; (I) and NIKi/Compound 31.

FIG. 8. A xenograft model for tamoxifen resistance. Growth kinetics of MCF-7 xenograft tumor cells expressing high endogenous APOBEC3B levels or depleted APOBEC3B levels due to transduction with non-specific shRNA or an APOBEC3B-specific shRNA, respectively (warm vs cool colors, respectively), n=5 animals/condition; data points represent mean tumor size+s.d. The knockdown of endogenous APOBEC3B suppresses the development of tamoxifen-resistant tumors.

FIG. 9. APOBEC3B upregulation by PMA is dependent on NF-κB-inducing kinase (NIK). (A) Representative dose response experiment for MCF10A cells treated with PMA following a 30-minute pretreatment with the indicated concentrations of NIK inhibitor (NIKi; Compound 31 from Li et al., 2013, Bioorganic & Medicinal Chemistry Letters 23:1238-1244). mRNA expression is reported as the mean of three independent RT-qPCR reactions normalized to TBP (error bars report SD from triplicate assays). (B) Representative dose response experiment in which A549 cells with a canonical NF-kB luciferase reporter are treated with TNF-α (canonical pathway inducer) or TNF-α with the indicated concentrations of NIK inhibitor (non-inhibitory in this assay) or parthenolide (Parth), which is a known canonical pathway inhibitor (Kwok et al., 2001, Chemistry & Biology 8, 759-766).

### DETAILED DESCRIPTION OF ILLUSTRATIVE EMBODIMENTS

This disclosure identifies a signal transduction pathway responsible for APOBEC3B upregulation, and several methods to inhibit this pathway and therefore stop APOBEC3B expression and genomic DNA mutagenesis. Accordingly, this disclosure provides methods of treating a subject having cancer or at risk of having cancer. The methods generally involve decreasing expression of APOBEC3B by administering to the subject an inhibitor of the PKC-NFκB signaling axis. Decreasing APOBEC3B expression, in combination with current treatment methods, can help suppress cancer mutagenesis, dampen tumor evolution, and/or decrease the probability of adverse outcomes, such as drug resistance and/or metastases. Stopping mutagenesis may also inhibit tumor evolution including subclonal outgrowths and enable more robust immune responses against clonal neoantigens, especially in combination with immune checkpoint inhibitors.

Somatic mutations are present in many forms of cancer. Mutations happen when DNA damage escapes repair. Established sources of mutation include, for example, ultraviolet light in skin cancer, tobacco carcinogens in lung cancer, and water-mediated deamination of methyl-cytosine as a function of age in many other cancers. A more recently discovered source of mutation is the plant-derived dietary supplement aristolochic acid, which causes A-to-T transversion mutations in liver and bladder cancers. Another source of mutation is the APOBEC family of DNA cytosine deaminases, which cause signature C-to-T transition and C-to-G transversion mutations in, for example, breast, head/neck, bladder, cervical, lung, ovarian, and other cancers. Many APOBEC mutational events are dispersed throughout the genome, and a minority of APOBEC mutational events can be found in dense strand-coordinated clusters termed kataegis.

Expression profiling and functional studies independently identified APOBEC as a major source of mutation in cancer. In particular, APOBEC3B is upregulated in breast and ovarian cancer cell lines and primary tumors. APOBEC3B is predominantly nuclear, and knockdown experiments demonstrated APOBEC3B-mediated DNA cytosine deaminase activity in cancer cell line extracts. Moreover, APOBEC3B mediates elevated levels of genomic uracil and increased mutation rates. APOBEC3B levels correlate with higher C-to-T and overall base substitution mutation loads. The biochemical preference of recombinant APOBEC3B deduced in vitro closely resembles the actual cytosine mutation bias in breast cancer as well as in several of the other tumor types listed above (i.e., strong bias toward 5′-TC dinucleotides).

Human cells have the potential to express up to seven distinct antiviral APOBEC3 enzymes. Each enzyme has a biochemical preference for deaminating cytosines in single-stranded DNA, but activity is strongly influenced by flanking bases at the −2, −1, and +1 positions relative to the target cytosine. APOBEC3B is the only family member obviously upregulated in the cancers listed above.

This disclosure describes APOBEC3B upregulation through the PKC-NFκB pathway. PKC activation by the diacylglycerol mimic PMA causes specific and dose-responsive increases in APOBEC3B mRNA, protein, and activity levels, which are strongly suppressed by PKC and NFκB inhibition. Induction correlates with RELB (but not RELA) recruitment to endogenous APOBEC3B implicating noncanonical NFκB signaling. Relevance to tumors is supported by PKC inhibitor-mediated APOBEC3B downregulation in multiple cancer cell lines. These data establish the first mechanistic link between APOBEC3B and a common signal transduction pathway. Thus, existing PKC-NFκB inhibitors can be repurposed to suppress cancer mutagenesis, dampen tumor evolution, and/or decrease the probability of adverse outcomes such as drug resistance and metastases.

### Specific Upregulation of APOBEC3B by PMA

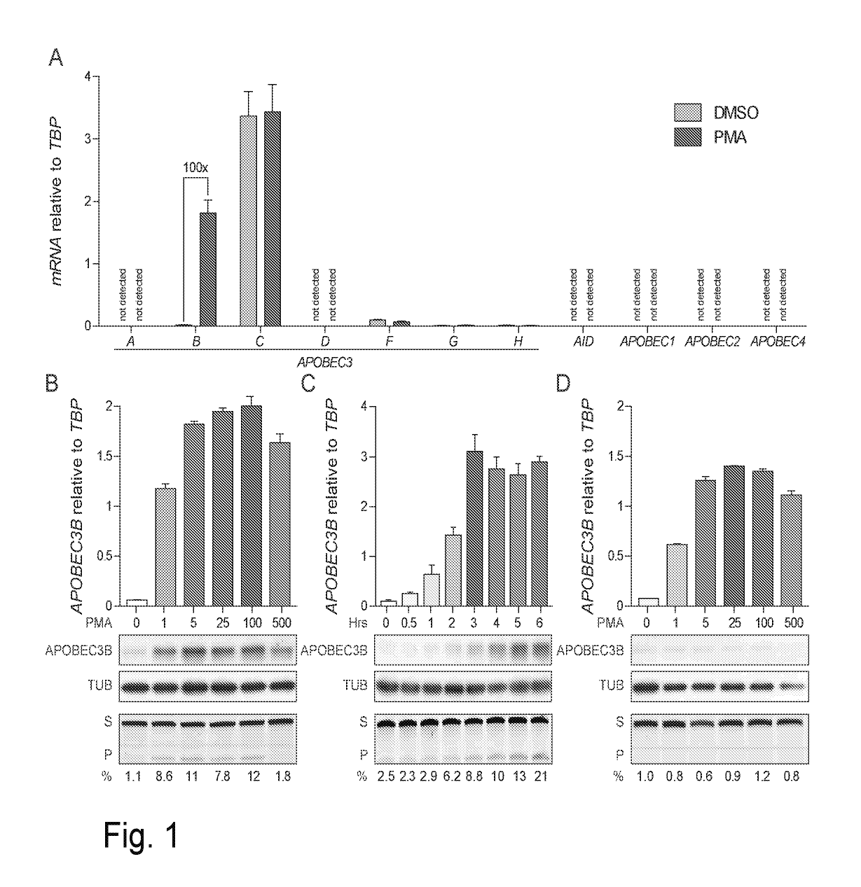

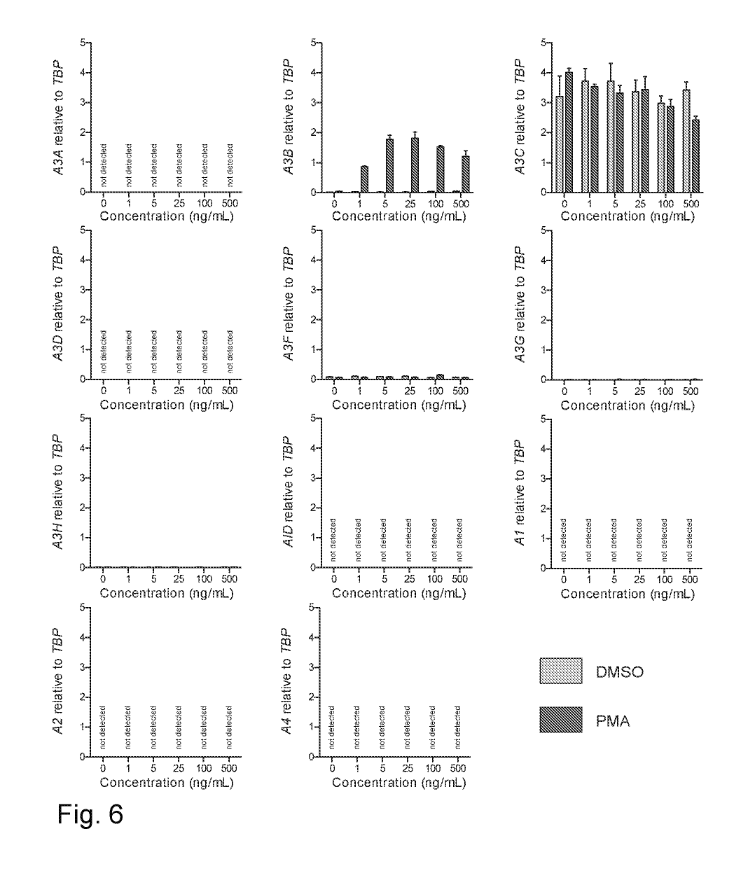

A panel of immortalized normal human epithelial cells lines and breast cancer cell lines was treated with PMA or equal amounts of DMSO as a negative control, and previously validated reverse transcription quantitative PCR (RT-qPCR) assays were used to measure mRNA levels of all eleven human APOBEC family members. APOBEC3B mRNA was induced specifically by PMA treatment of several lines including the immortalized normal breast epithelial cell line MCF10A (FIG. 1A and FIG. 6). Under standard cell culture conditions, MCF10A expresses low levels of APOBEC3B and APOBEC3F, even lower levels of APOBEC3G and APOBEC3H, high levels of APOBEC3C, and undetectable levels of all other APOBEC family members. PMA treatment caused a specific 100-fold upregulation of APOBEC3B mRNA, with no detectable changes in the expression levels of any other APOBEC family members (FIG. 1A and FIG. 6).

APOBEC3B was induced with as little as 1 ng/mL PMA, and its induction was dose responsive and near maximal at 25 ng/mL PMA (FIG. 1B, histogram). APOBEC3B mRNA levels correlated with a rise in steady-state protein levels as measured by immunoblotting with a rabbit anti-APOBEC3B monoclonal antibody (described in U.S. Provisional Patent Application No. 62/186,109. filed Jun. 29, 2015) and with enzymatic activity as measured by a gel-based single-stranded DNA cytosine deamination assay (FIG. 1B). Moreover, significant APOBEC3B mRNA induction was detected 30 minutes after PMA treatment and maximal levels were observed by three hours post-treatment (FIG. 1C, histogram). APOBEC3B protein and activity levels lagged shortly behind mRNA levels and persisted through the duration of the time course (FIG. 1C, immunoblot and polyacrylamide gel). APOBEC3B upregulation may be a direct result of signal transduction as the kinetics of upregulation were not affected by simultaneously treating cells with the protein translation inhibitor cyclohexamide (FIG. 1D). Altogether, these data demonstrate that APOBEC3B is strongly and specifically upregulated by a PMA-induced signal transduction mechanism in the immortalized normal breast epithelial cell line MCF10A. Notably, upregulation can be as high as 100-fold and this maximal level of APOBEC3B mRNA is consistent with that observed in many different tumor types including, for example, a large fraction of breast and ovarian cancers—e.g., mRNA levels 2-fold to 5-fold higher than those of the constitutively expressed housekeeping gene TBP.

### PKC is Involved in APOBEC3B Induction by PMA

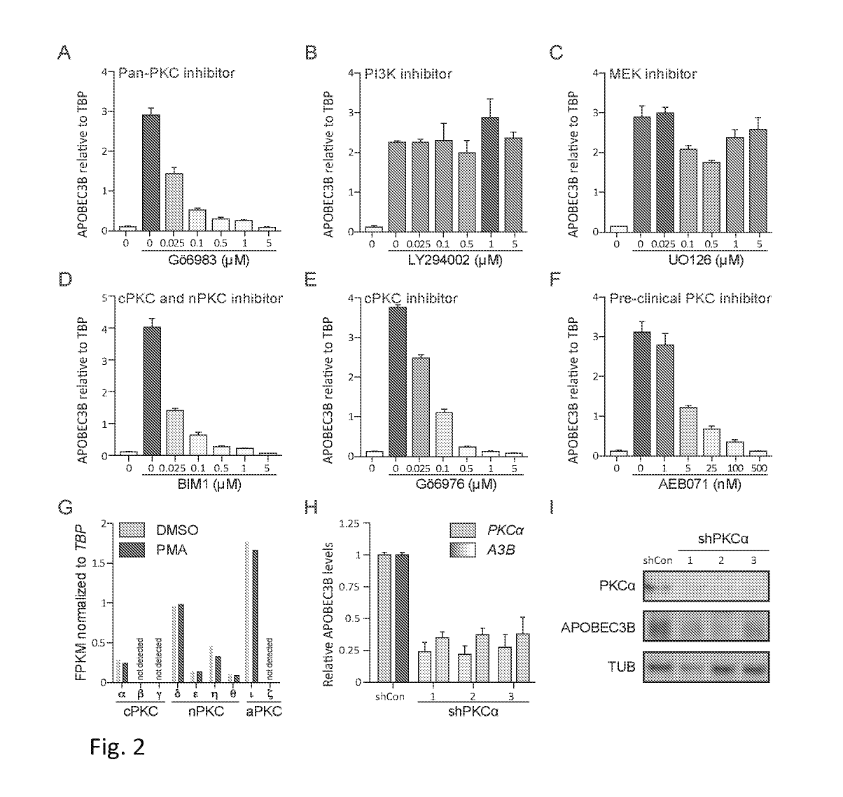

PMA is a well-known agonist of PKC, but it also affects other cellular processes. To determine whether APOBEC3B induction by PMA occurs through PKC signal transduction or an alternative mechanism, we leveraged a panel of existing PKC inhibitors that each vary with respect to class selectivity. MCF10A cells were pre-treated for 30 minutes with varying concentrations of the pan-PKC inhibitor Gö6983 (Gschwendt et al., FEBS Lett. 392:77-80, 1996) and then treated for six hours with PMA (25 ng/mL). In comparison to strong APOBEC3B upregulation observed with PMA treatment alone, pretreatment with Gö6983 caused a dose responsive suppression of APOBEC3B induction (FIG. 2A). APOBEC3B was suppressed to background levels with 5 μM Gö6983, as well as higher concentrations (FIG. 2A). No morphological defects or viability issues were observed at these low concentrations of Gö6983 (data not shown). As additional controls, MCF10A cells were pretreated in parallel with the phosphoinositol 3 kinase (PI3K) inhibitor, LY294002, and the mitogen-activated protein kinase kinase (MEK) inhibitor, U0126, prior to PMA induction (FIGS. 2B and 2C). In both instances, no suppression of APOBEC3B upregulation was observed. Taken together, these data indicated that the PKC pathway regulates endogenous APOBEC3B expression in the MCF10A breast epithelial cell line, and the PI3K and MEK pathways are unlikely to be involved.

Human cells can express up to nine different PKC genes. The resulting PKC proteins (conventionally called isoforms) are divisible into three classes based primarily on activation mechanism: classical PKC (cPKC) isoforms require both DAG and increased levels of intracellular calcium, novel PKC (nPKC) isoforms require only DAG, and atypical PKC (aPKC) isoforms are activated by other signals. To determine which class of PKC isoforms is responsible for APOBEC3B upregulation, one can use two additional inhibitors known to have potency similar to Gö6983, but greater selectivity for certain PKC classes. First, MCF10A cells were pretreated with bisindolylmaleimide-1 (BIM-1), which inhibits both cPKC isoforms and nPKC isoforms, and then induced with optimal PMA concentrations. A nearly identical dose dependent suppression of APOBEC3B induction was observed (FIG. 2D). Second, MCF10A cells were pretreated with Gö6976, which is an inhibitor of cPKC isoforms. The dose responsiveness of APOBEC3B repression was again similar to Gö6983 (FIG. 2E). Taken together, these chemical inhibition data strongly implicated a cPKC isoform in APOBEC3B induction by PMA.

AEB071 selectively inhibits cPKC and nPKC isoforms AEB071 has shown results in preclinical studies and phase I clinical trials for treatment of uveal melanoma. MCF10A cells were pretreated with AEB071 to determine whether AEB071 produces a similar reductive effect on PMA induced APOBEC3B expression as the above PKC inhibitors. Indeed, a clear dose dependent response was observed and, importantly, AEB071 caused a complete suppression of APOBEC3B expression at 500 nM, which is approximately 10-fold more potent than Gö6983, BIM-1, or Gö6983, consistent with reported lower IC₅₀ values for this molecule (FIG. 2F).

RNAseq data revealed that PKCα (PRKCA) is the only cPKC isoform expressed in MCF10A cells (FIG. 2G). PKCα mRNA levels were unchanged by PMA treatment, consistent with a mechanism by which PMA signals through pre-existing PKCα to ultimately stimulate APOBEC3B transcription (FIG. 2G). PKCα expression was depleted using three independent shRNA-encoding lentiviral constructs. In each case, PKCα knockdown resulted in a corresponding reduction in the level of APOBEC3B mRNA induced by PMA (FIG. 2H). Immunoblots confirmed PKCα knockdown and proportional reductions in APOBEC3B (FIG. 21). Altogether, the pharmacologic and genetic approaches provide a strong case for PKCα as the predominant PKC isoform driving PMA-mediated upregulation of APOBEC3B.

NFκB is Involved with APOBEC3B Induction by PMA

We next investigated which downstream transcription factor is responsible for driving APOBEC3B upregulation in response to PMA. PKC signals through several different transcription factors, including ERK, JNK, NFκB, and others. We therefore started at the DNA level and examined the APOBEC3B promoter region for binding sites of known PKC-regulated transcription factors. These in silico analyses revealed several NFκB binding sites within 2.5 kb of the APOBEC3B transcriptional start site (5′-GGRRNNYYCC-3′; SEQ ID NO:1).

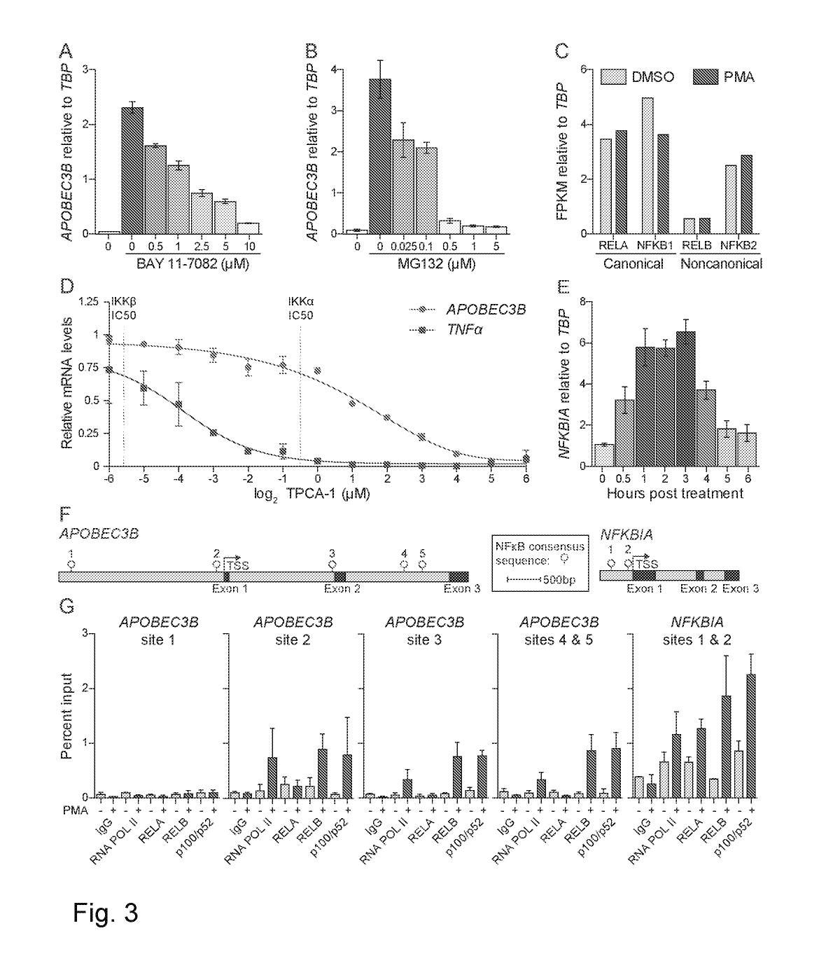

To test for a mechanistic link between NFκB and APOBEC3B transcription, we pretreated MCF10A cells with varying amounts of BAY 11-7082, which is an NFκB inhibitor that acts by inhibiting upstream IκB kinases (IKKs), and then added PMA at concentrations effective for APOBEC3B induction. BAY 11-7082 caused strong dose-responsive drops in APOBEC3B induction by PMA treatment (FIG. 3A).

The canonical and noncanonical NFκB signaling pathways involve proteasome-mediated degradation of IκB and p100, respectively, for efficient signal transduction. Therefore, we blocked degradation of these proteins by pretreating MCF10A cells with a titration of the proteasome inhibitor, MG132, prior to PMA stimulation. Under these conditions, APOBEC3B expression decreased in a dose dependent manner in response to MG132 treatment (FIG. 3B), indicating that the pathway of interest requires protein degradation by the proteasome for productive signal transduction.

RNAseq data revealed that MCF10A expresses both the canonical NFκB components, RELA and NKB1, and the non-canonical NFκB components, RELB and NFKB2, and levels of these mRNAs are unaffected by PMA treatment (FIG. 3C). Canonical signaling is known to require IKKβ, whereas non-canonical NFκB signaling is strictly dependent on IKKα-catalyzed phosphorylation of p100. To distinguish between these pathways, MCF10A cells were pretreated with a titration of TPCA-1 concentrations spanning the IC50 values of both proteins, and then PMA was used to induce APOBEC3B upregulation. TPCA-1 exhibits a 22-fold selectivity for IKKβ (canonical) over IKKα (non-canonical). APOBEC3B expression was inhibited closer to the reported IC₅₀ of IKKα, consistent with involvement of the non-canonical NFκB pathway (FIG. 3D). As an additional control, TNFα, which is regulated by the canonical pathway, was analyzed. TNFα expression was inhibited by much lower concentrations of TCPA-1, confirming the differential selectively of this compound and further implicating the non-canonical NF□B pathway (FIG. 3D).

### RELB is Recruited to the APOBEC3B Promoter Region in Response to PMA

We next performed a series of chromatin immunoprecipitation (ChIP) experiments to determine whether the canonical or noncanonical NFκB pathway is responsible for upregulating APOBEC3B. Primer sets were designed for each of the predicted NFκB binding sites near the APOBEC3B transcriptional start site, as well as a control set in the promoter region of NFKIB (FIG. 3E). ChIP was performed for RELA, RELB, RNA POL II (positive control), and isotype matched IgG (negative control) RELA, RELB, and RNA POL II were all bound to the NFKIB promoter following PMA treatment (FIG. 3F and FIG. 3G). In addition, we found RNA POL II strongly bound to the APOBEC3B gene near the transcriptional start site (FIG. 3F and FIG. 3G). We also found RELB bound both near the transcriptional start site and at sites 4 and 5, which are located in intron 2 and too close together to be distinguished by this procedure. Binding also may occur at lower levels at site 3 in intron 1, but the IgG signal was too high to distinguish background from actual binding. These ChIP data strongly implicate the noncanonical NFκB pathway, specifically RELB (and not RELA), in directly inducing APOBEC3B transcription in response to PMA activation of PKC.

### The NF-κB Inhibitory Kinase, NIK, is Required for PMA-Induced APOBEC3B Upregulation.

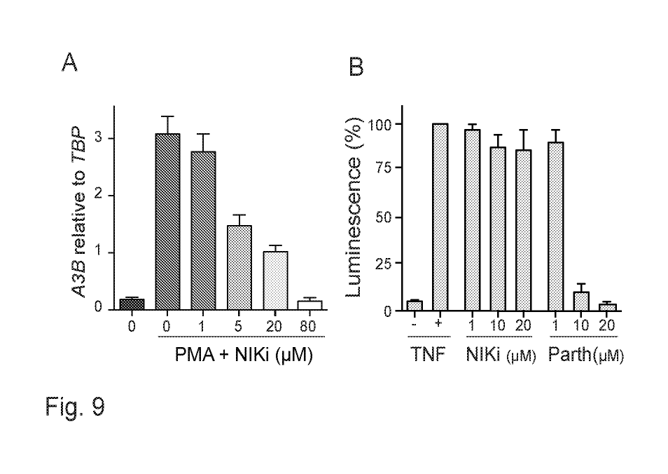

A published NIK inhibitor (NIKi, Compound 31 from Li et al., 2013, Bioorganic & Medicinal Chemistry Letters 23:1238-1244) blocks APOBEC3B upregulation in the breast epithelial cell line MCF10A, and does not affect canonical NF-κB gene expression (FIG. 9). MCF10A cells were pretreated 30 minutes with varying concentrations of NIKi, PMA was used to induce APOBEC3B and six hours later mRNA expression was quantified by RT-qPCR. As above for several PKC inhibitors, NIKi caused a strong dose responsive suppression of APOBEC3B expression.

### Endogenous APOBEC3B Expression is Mediated by the PKC-NFκB Axis in Multiple Cancer Cell Lines

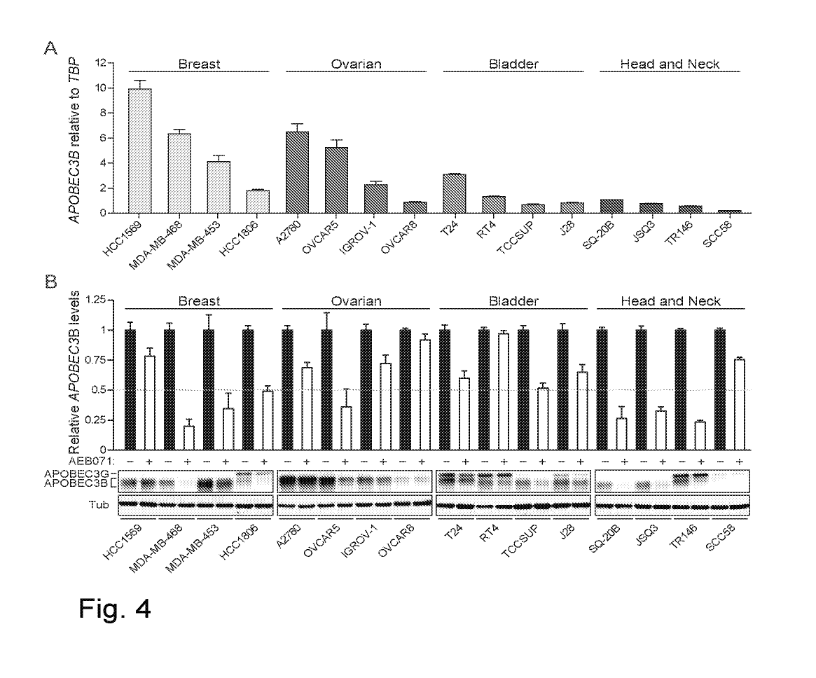

Four breast cancer cell lines, four ovarian cancer cell lines, four bladder cancer cell lines, and four head/neck cancer cell lines were analyzed to determine whether the constitutively high levels of endogenous APOBEC3B observed in many human cancer cell lines occurs through the PKC pathway. The selected cell lines expressed a 10-fold range of endogenous APOBEC3B mRNA levels (FIG. 4A).

Each line was treated for 48 hours with 10 μM AEB071, and then APOBEC3B mRNA and protein levels were quantified by RT-qPCR and immunoblotting. No effects on the cell cycle or cell viability were observed (data not shown). This is important since higher concentrations of AEB071 are known to cause cell cycle perturbations and apoptosis in certain cell types. APOBEC3B mRNA levels were reduced by more than half in 7/16 cell lines, including the breast cancer cell lines MDA-MB-468, MDA-MB-453, and HCC1806, the ovarian cancer cell line OVCAR5, and the head/neck lines SQ-20B, JSQ3, and TR146 (FIG. 4B, histogram). Changes of protein levels largely mirrored the mRNA results (FIG. 4B, immunoblot). Together, these data demonstrate that the PKC axis is responsible for the constitutive upregulation of endogenous APOBEC3B in a variety of cancer cell lines representing multiple distinct cancer types.

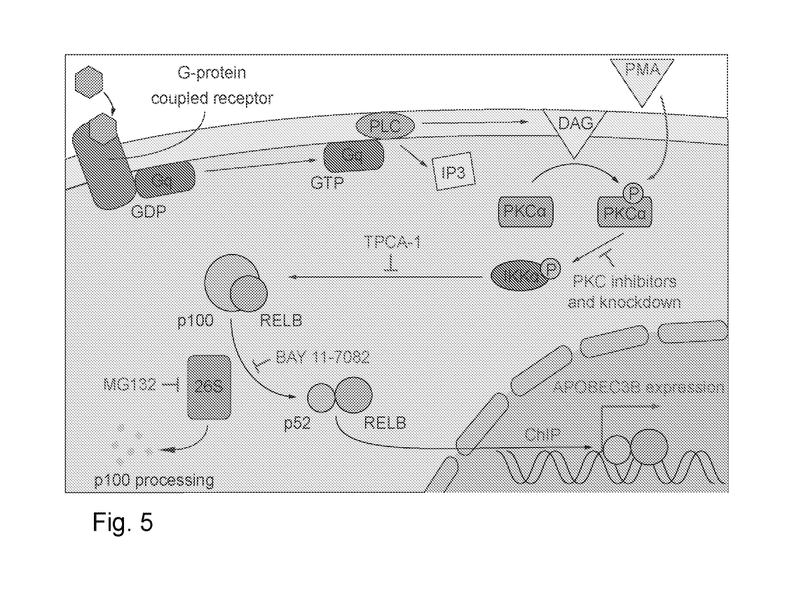

The studies described above are the first to demonstrate that the PKC-NFκB pathway is responsible for inducing ABOBEC3B expression in breast, ovarian, bladder, and head/neck cancers. A series of experiments using the immortalized normal breast epithelial cell line MCF10A showed that the diacylglycerol analog PMA activates PKCα, which then signals through the non-canonical NFκB pathway and results in the recruitment of RELB to the APOBEC3B gene and its transcriptional activation (FIG. 5). This mechanism appears specific to APOBEC3B, as expression of the related APOBEC family members is not affected. This specificity is consistent with APOBEC3B being the only DNA deaminase family member upregulated in these and other cancer types in comparison to normal tissues. Moreover, PKC inhibitor studies with cancer cell lines indicated that the PKC-NFκB pathway may be responsible for the constitutively high levels of APOBEC3B documented previously in a large proportion of breast, ovarian, bladder, head/neck, and other cancers.

APOBEC3B overexpression and mutation signatures in cervical and head/neck cancers suggest that HPV infection might trigger an innate immune response that includes DNA deaminase upregulation. Also, infection by high-risk HPV types (not low-risk types) causes the specific upregulation of APOBEC3B, suggesting that this is not simply a gratuitous innate immune response to viral infection. Moreover, the E6 oncoprotein from high-risk types (again, not low risk) can be, all by itself, sufficient to trigger APOBEC3B upregulation. Also, the E7 oncoprotein may contribute to APOBEC3B upregulation. The mutator phenotype induced by HPV infection is likely fueling tumor evolution as the pattern of PI3K-activating mutations in HPV-positive tumors is completely biased toward cytosine mutations in APOBEC signature motifs in the helical domain of the kinase, whereas the pattern in HPV-negative tumors is split between the helical and kinase domains of the enzyme. While HPV-mediated upregulation of APOBEC3B predominantly impacts cervical and a proportion of head/neck and bladder carcinomas, other tumor types may be susceptible to the mechanism described here.

Although PKC mutations are rare in cancer, altered expression of several PKC isoforms is observed and associated with poor clinical outcomes. In addition, mutations in GNAQ and GNA11 occur in approximately half of all uveal melanoma samples (illustrated as Gq in FIG. 5). Inhibition of PKC in these uveal tumors leads to clinical benefits. It is possible that part of these encouraging clinical responses is due to downregulating APOBEC3B and decreasing each tumor's capacity to evolve and yield potentially detrimental mutations. Based on substantive prior work from our lab and others demonstrating a major role for APOBEC3B in cancer mutagenesis and correlating high levels of APOBEC3B with poor prognoses for ER-positive breast cancers, together with the studies presented here, existing inhibitors of the PKC-NFκB axis (such as, for example, AEB071) may be repurposed to treat primary tumors in combination with existing therapies and help prevent detrimental outcomes such as drug resistance and metastases.

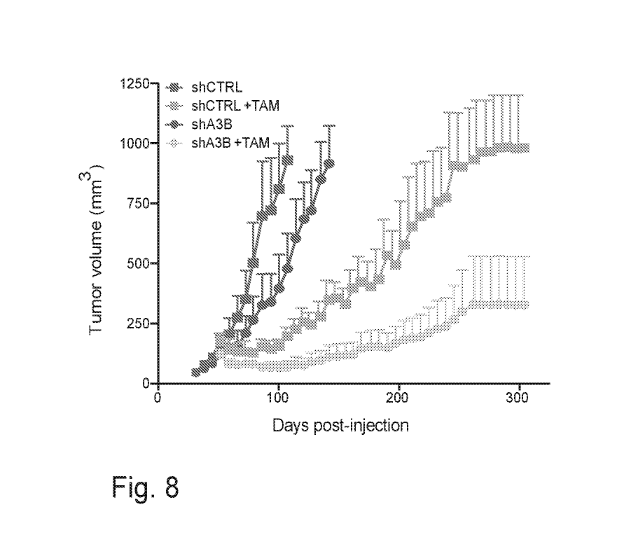

Thus, this disclosure generally describes methods that involve using a PKC-NFκB axis inhibitor to decrease expression of APOBEC3B and thereby control the mutational potential of tumor cells. In some embodiments, these methods can be practiced in the context of an anti-tumor therapy that involves administering a PKC-NFκB axis inhibitor to a subject in need of therapy that involves decreasing expression of APOBEC3B. A decrease in APOBEC3B expression can decrease the mutational potential of tumor cells and, therefore, decrease the severity and/or extent of growth and/or metastasis of the tumor. Such an APOBEC3B inhibitory therapy may be especially effective, for example, in combination with a targeted therapy such as tamoxifen to inhibit the development of resistance mutations (FIG. 8). It also may be effective, for example, in the context of an immunotherapy by inhibiting the evolution of new neoantigens and thereby helping the immune response to focus on responding to clonal tumor antigens present in all tumor cells, since ongoing tumor evolution may “distract” the immune response from the clonal antigens. Thus, an APOBEC3B inhibitor may stop this process and in turn promote tumor clearance by enabling more robust immune responses.

Thus, in one aspect, this disclosure describes methods of treating a subject having or at risk of having a tumor. Generally, the method includes administering to a person having or at risk of having a tumor an effective amount of a PKC-NFκB axis inhibitor. As used herein, “treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment can refer to therapy initiated after the condition manifests in a subject, while “prophylactic” treatment can refer to therapy initiated before a condition manifests in a subject. As used herein, “symptom” refers to any subjective evidence of disease or of a patient's condition; “sign” or “clinical sign” refers to an objective physical finding relating to a particular condition capable of being found by one other than the patient, and includes molecular and cellular signs such as, for example, decreased APOBEC3B expression. As used herein, “ameliorate” refers to any reduction in the extent, severity, frequency, and/or likelihood of a symptom or clinical sign characteristic of a particular condition.

Prophylactic treatment typically involves treating a subject before a condition manifests in a subject. Accordingly, prophylactic may be initiated in a subject that is “at risk” for developing the condition. A subject “at risk” refers to a subject that may or may not actually have or possess the condition or manifest any indication (e.g., a symptom or clinical sign) of the condition. Thus, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition. In another aspect, the method can include confirming the presence of APOBEC3B in cells of the tumor and administering to the subject an amount of a PKC-NFκB axis inhibitor effective to decrease the level of APOBEC3B in the tumor cells.

The presence of APOBEC3B in the tumor cells can be confirmed by any suitable method. Exemplary methods include, for example, suitable forms of chromatography, electrophoresis, and/or immunoassays. In some cases, an immunoassay can employ an APOBEC3-specific antibody such as, for example, an antibody described in U.S. Provisional Patent Application No. 62/186,109, filed Jun. 29, 2015. In some embodiments, the presence of APOBEC3B in the cells of the tumor is assayed by RT-qPCR, detecting an APOBEC3B signature mutation through DNA sequencing, or detecting the protein itself using an APOBEC3B-specific antibody.

As used herein “antibody”—when not preceded by a definite or indefinite article—can be used generically to refer to any preparation that includes at least one molecular species of immunoglobulin or a fragment (e.g., scFv, Fab, F(ab′)₂ or Fv or other modified fragment) thereof. Therefore, “antibody” can generically include one or more monoclonal antibodies and/or a polyclonal antibody preparation. As used herein, “specific” and variations thereof (e.g., “APOBEC3B-specific”) refer to having a differential or a non-general affinity, to any degree, for a particular target. In some embodiments, the APOBEC3-specific antibody can be antibody described in U.S. Provisional Patent Application No. 62/186,109, filed Jun. 29, 2015.

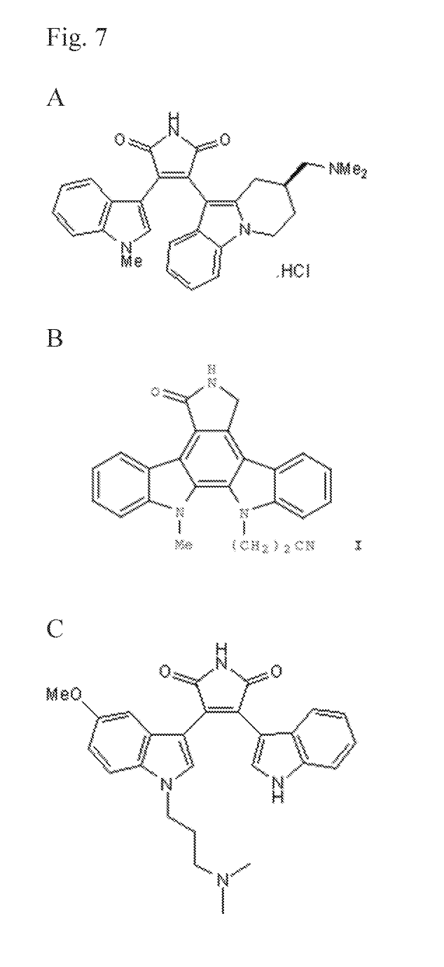

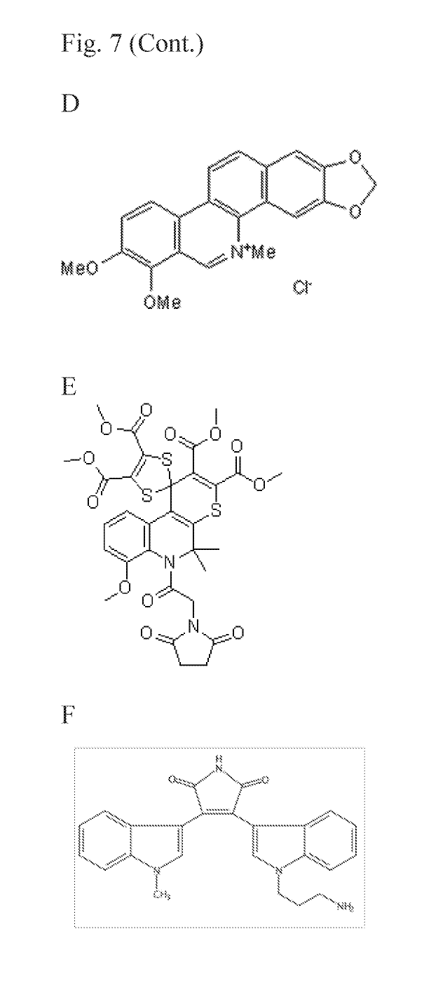

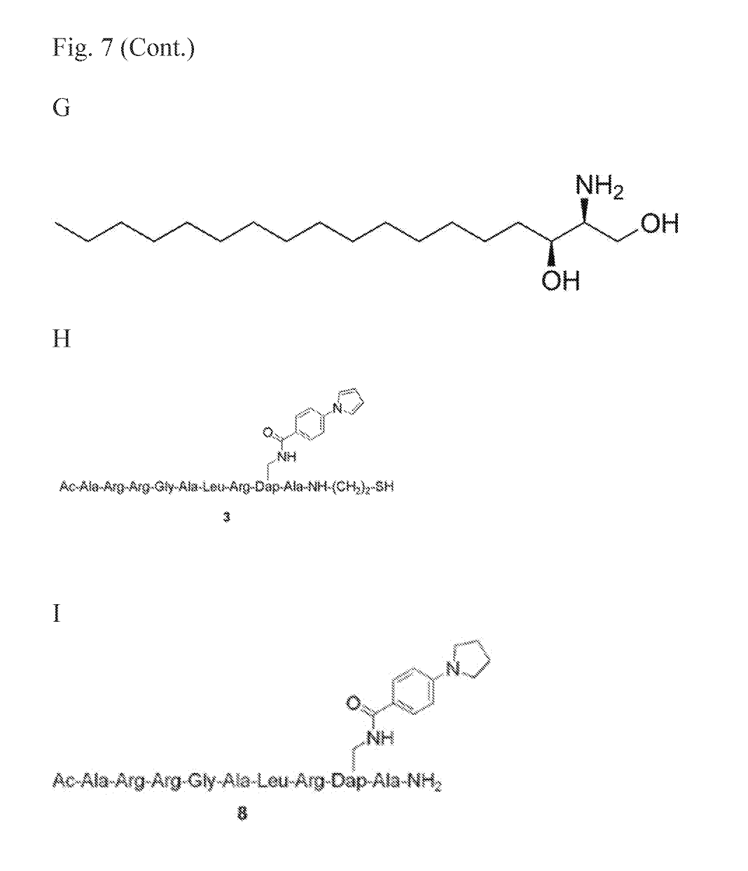

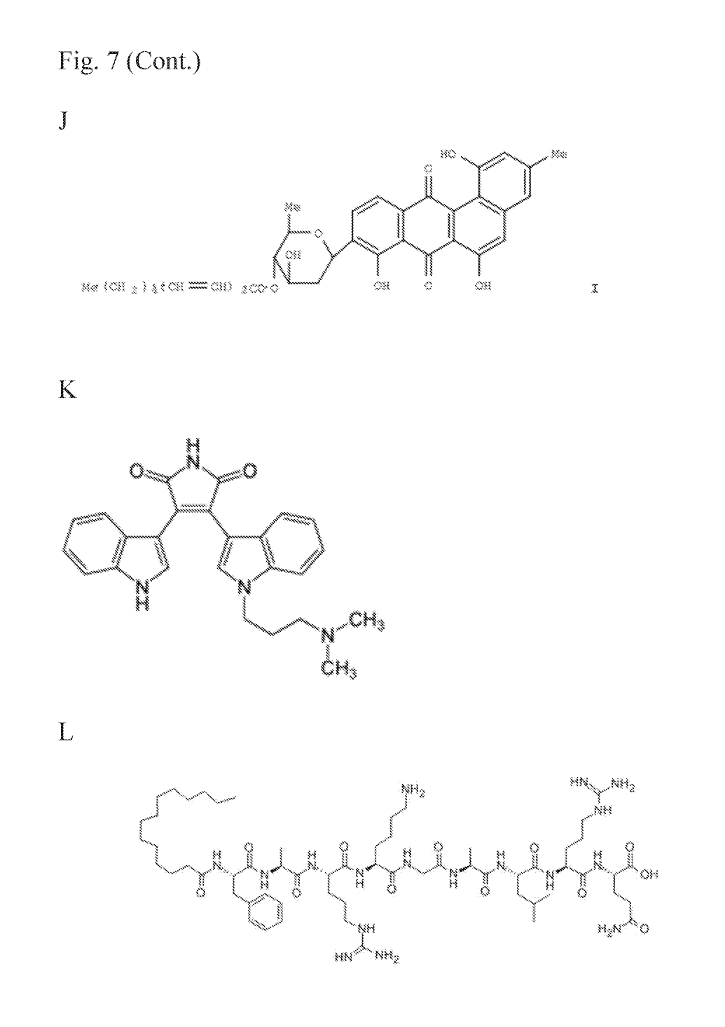

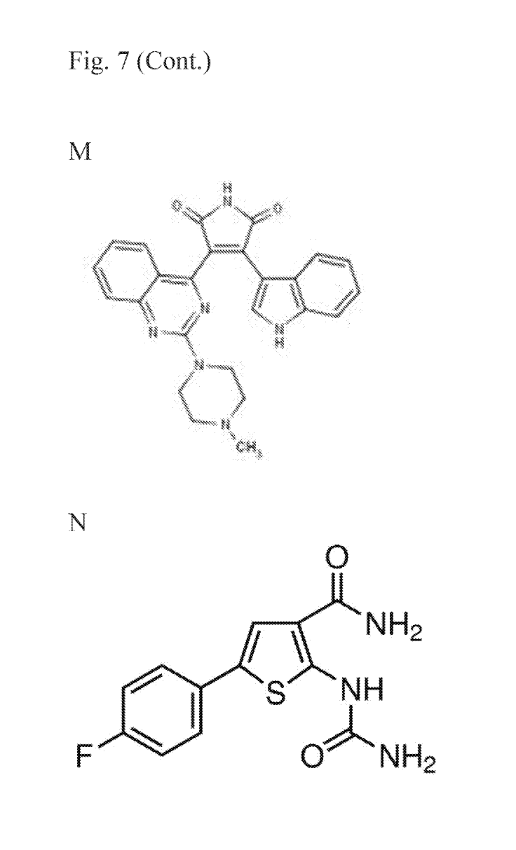

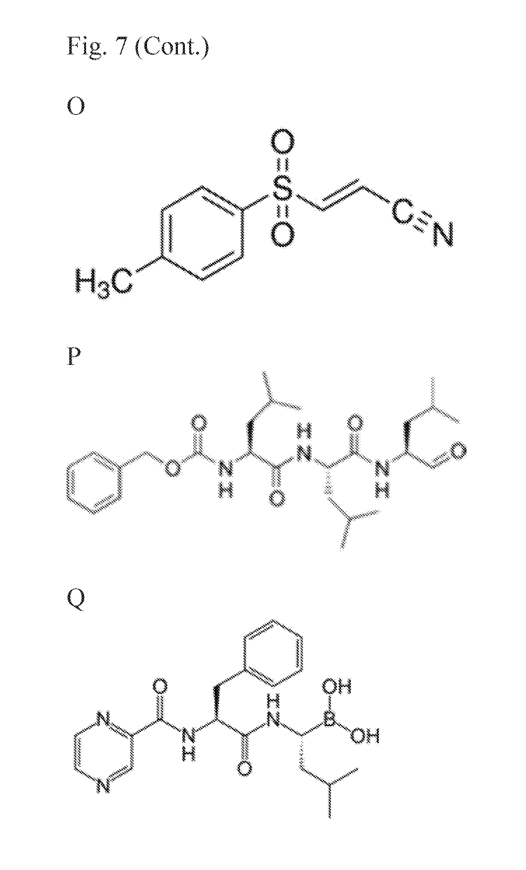

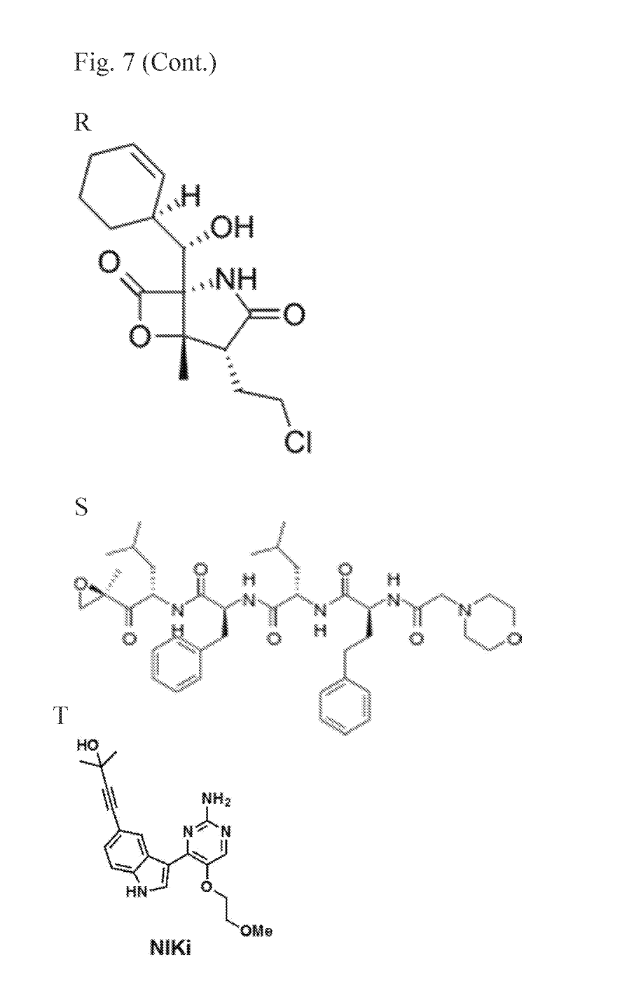

The PKC-NFκB axis inhibitor can be any compound or composition that inhibits cell signaling within the PKC-NFκB axis. Exemplary PKC-NFκB axis inhibitors include, for example, Gö6983 (FIG. 7C), Gö6976 (FIG. 7B), MT477 (FIG. 7E), RO 32-0432 (FIG. 7A), chelerythrine (FIG. 7D), RO 31-7549 (FIG. 7F), safingol (FIG. 7G), Compound 3 (FIG. 7H) and Compound 8 (FIG. 71) described in Lee et al. (Bioorg. Med. Chem. Lett. 15:2271-2274 (2005)), PKC inhibitors described in U.S. Patent Application Publication No. US 2007/0293525 A1, balmoralmycin (I) (FIG. 7J), bisindolylmaleimides (e.g., bisindolylmaleimide-1, shown in FIG. 7K), aprinocarsen (ISIS 3521; Yuen et al., 1999, Clinical Cancer Research 5(11):3357-3363), myr-FARKGALRQ (FIG. 7L), sotrastaurin (AEB071) (FIG. 7M), TPCA-1 (FIG. 7N), BAY 11-7082 (FIG. 7O), MG132 (FIG. 7P), bortezomib (FIG. 7Q), salinosporamide A (FIG. 7R), carfilzomib (FIG. 7S), or a PKC inhibitor such as 1-(5-isoquinolinesulfonyl)-2-methylpiperazine or NIKi/Compound 31 (FIG. 7T).

Alternative PKC-NFκB axis inhibitors can include, for example, a RelB-p52 inhibitor. RelB-p52 is a protein heterodimer that binds DNA and activates APOBEC3B expression. One exemplary RelB-p52 inhibitor is SN52 (AAVALLPAVLLALLAPVQRKRRKALP; SEQ ID NO:3), which blocks nuclear translocation of RelB-p52. Other RelB-p52 inhibitors can include, for example, variants of SN52 (Yu et al., 2008, Mol. Cancer Ther. 7(8):2367-2376) or a DNA decoy targeted to the RelB-p52 DNA-binding sequence and, therefore, inhibit APOBEC3B expression. Such a DNA decoy can compete with RelB-p52 for DNA binding, and therefore, inhibit APOBEC3B expression by competitive sequestration of RelB-p52 from its genomic binding site.

Alternative PKC-NFκB axis inhibitors can include, for example, a PKCβ-selective small molecule inhibitor such as, for example enzastaurin or ruboxistaurin.

Still other PKC-NFκB axis inhibitors can include, for example, inhibitors of the IKK kinase family (e.g., IKK-α, IKK-γ) and NIK. Exemplary IKK inhibitors include compounds described in, for example, Llona-Minguez et al., 2013, Pharm. Pat. Analyst 2(4):481-498. An exemplary NIK inhibitor is NIKi/Compound 31 (FIG. 7T).

The PKC-NFκB axis inhibitor can be administered to a subject having or at risk of having a tumor such as, for example, a tumor resulting from acute lymphoblastic leukemia (ALL), bladder cancer, breast cancer, cervical cancer, chondrosarcoma, chronic lymphocytic leukemia (CLL), esophageal cancer, head and neck cancer, kidney cancer, lung cancer, B cell lymphoma, melanoma, myeloma, osteosarcoma, ovarian cancer, pancreatic cancer, stomach cancer, thyroid cancer, uterine cancer, and uveal cancer. As used herein, the term “tumor” refers to a general state of neoplasia and does not necessarily connote a solid mass. Thus, as used herein, a tumor may be characterized as solid or as liquid. Generally, a solid tumor involves a solid mass of neoplastic cells. Generally, a liquid tumor involves neoplasias of the blood, bone marrow or the lymphatic system and do not necessarily form a solid mass.

A PKC-NFκB axis inhibitor may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PKC-NFκB axis inhibitor without causing any undesirable biological effects or interacting in a deleterious manner with any of the other components of the pharmaceutical composition in which it is contained.

A PKC-NFκB axis inhibitor may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), topical (e.g., intranasal, intrapulmonary, intramammary, intravaginal, intrauterine, intradermal, transcutaneous, rectally, etc.) or intratumoral. A pharmaceutical composition may be formulated for administration to a mucosal surface, such as by administration to, for example, the nasal or respiratory mucosa (e.g., by spray or aerosol).

A pharmaceutical composition may be delivered using any suitable drug delivery device or technology. In some cases, a pharmaceutical composition may be administered for systemic exposure. In other embodiments, the pharmaceutical composition may be administered for local or targeted exposure of a particular body compartment, tissue, or tumor. In some cases, a drug delivery technology can control the kinetics of release of the pharmaceutical composition to provide, for example, a pulsatile profile, a sustained or continuous profile, a delayed onset profile, or some combination of these profiles. One exemplary drug delivery approach includes liposomes loaded with a pharmaceutical composition. The liposomes can be functionalized with aptamers, peptides, and/or segments of DNA or RNA for targeting delivery to and uptake into a particular cell type or tumor. Similarly, hollow spherical nucleic acids (SNA) can carry a pharmaceutical composition into a cell through the use of ordered and functionalized oligonucleotides placed on the surface of the SNA. Degradable and non-degradable polymeric particles (e.g., microparticles and/or nanoparticles) can be loaded with the pharmaceutical composition for systemic or local drug distribution. In some embodiments, the pharmaceutical composition may be delivered using polymer micelles. Iontophoresis, ultrasound, and other forms of energy can be used to increase the permeability of a drug into a tissue or cell. In some embodiments, the pharmaceutical composition may be delivered using implantable drug delivery device. In some of these embodiments, an implantable device such as, for example, a degradable polymer depot can have a short duration of action (e.g., hours to weeks). In other embodiments, an implantable device such as, for example, a pump or a microdevice can be implanted to deliver a pharmaceutic composition over a longer (e.g., months, years, or permanently) period of time. Many longer term and permanent implants may be programmed to deliver a drug at a particular time or with a specific kinetic profile. In addition, such devices can usually be refilled with the pharmaceutical composition from time to time. One or more PKC-NFκB axis inhibitors can be delivered by any one or any combination of drug delivery techniques.

Thus, a PKC-NFκB axis inhibitor may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PKC-NFκB axis inhibitor into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of PKC-NFκB axis inhibitor administered can vary depending on various factors including, but not limited to, the specific PKC-NFκB axis inhibitor, the weight, physical condition, and/or age of the subject, and/or the route of administration. Thus, the absolute weight of PKC-NFκB axis inhibitor included in a given unit dosage form can vary widely, and depends upon factors such as the species, age, weight and physical condition of the subject, and/or the method of administration. Accordingly, it is not practical to set forth generally the amount that constitutes an amount of PKC-NFκB axis inhibitor effective for all possible applications. Those of ordinary skill in the art, however, can readily determine the appropriate amount with due consideration of such factors. For example, certain PKC-NFκB axis inhibitors may be administered at the same dose and frequency for which the drug has received regulatory approval. In other cases, certain PKC-NFκB axis inhibitors may be administered at the same dose and frequency at which the drug is being evaluated in clinical or preclinical studies. One can alter the dosages and/or frequency as needed to achieve a desired level of APOBEC3B. Thus, one can use standard/known dosing regimens and/or customize dosing as needed.

In some embodiments, the method can include administering sufficient PKC-NFκB axis inhibitor to provide a dose of, for example, from about 100 ng/kg to about 50 mg/kg to the subject, although in some embodiments the methods may be performed by administering PKC-NFκB axis inhibitor in a dose outside this range. In some of these embodiments, the method includes administering sufficient PKC-NFκB axis inhibitor to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184. In some embodiments, the method can include administering sufficient PKC-NFκB axis inhibitor to provide a dose of, for example, from about 0.01 mg/m² to about 10 mg/m².

In some embodiments, PKC-NFκB axis inhibitor may be administered, for example, from a single dose to multiple doses per week, although in some embodiments the method can be performed by administering PKC-NFκB axis inhibitor at a frequency outside this range. In certain embodiments, PKC-NFκB axis inhibitor may be administered from about once per month to about five times per week.

In some embodiments, the PKC-NFκB axis inhibitor can be co-administered with a second therapy. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, resection of at least a portion of a solid tumor, etc.) persists at the treatment site until one or more additional components are administered to the treatment site. Thus, a co-administered combination can, in certain circumstances, include components that never exist in a chemical mixture with one another. In this context, therapy that includes administering a PKC-NFκB axis inhibitor can be concurrent with another anti-tumor therapy. In other embodiments, therapy that includes administering a PKC-NFκB axis inhibitor can be provided before or after a treatment regimen that includes another anti-tumor therapy. Exemplary anti-tumor therapies include, for example, chemotherapy, targeted therapy, immunotherapy, radiotherapy, or palliative care.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Cell Lines

MCF10A (ATCC CRL-10317), HCC1569 (ATCC CRL-2330, and MDA-MB-468 (ATCC HTB-132) were purchased from the American Type Culture Collection (ATCC) and cultured as recommended. A2780 and OVCARS were obtained from Dr. Scott Kaufmann (Mayo Clinic, Rochester, Minn.) and cultured as reported (Leonard et al., 2013, Cancer Res 73(24):7222-7231). SQ20B and JSQ3 were obtained from Dr. Mark Herzberg (University of Minnesota-Twin Cities, Minneapolis, Minn.) and cultured at 37° C. with 5% CO₂ in DMEM/F12 with 10% fetal bovine serum, penicillin, streptomycin, and 400 ng/mL hydrocortisone. These and other cell lines are listed in Table 1.

TABLE 1

| Cell Line | Type | Growth Conditions^{a} | Source |
|---|---|---|---|
| 293T | Fibroblast | DMEM, 10% FBS, Pen-Strep | Dr. Michael Malim |
| HeLa | Cervical Cancer | RPMI, 10% FBS, Pen-Strep | |
| MCF7L | Breast Cancer | IMEM, 5% FBS, Pen-Strep, 11.2 5 nM | Dr. Doug Yee, |
| | | human insulin | University of |
| | | | Minnesota |
| N/TERT | Keratinocyte | K-SFM, Pen-Strep, 0.3 mM CaCl₂, 0.2 ng/ml | Dr. Peter Howley, |
| | | EGF, 25 μg/ml BPE | Harvard University |
| NIKS | Keratinocyte | E media, 5% FBS, Pen-Strep, 24 μg/ml | Dr. Paul Lambert, |
| | | adenine, 8.4 μg/ml cholera toxin, 10 ng/ml | University of |
| | | EGF, 400 ng/ml hydrocortisone, 5 μg/ml | Wisconsin, Madison |
| | | insulin | |
| MCF10A | Epithelial | MEGM without gentimicin, Pen-Strep, | ATCC CRL-10317 |
| | | 10 μg/ml cholera toxin | |
| HCC1569 | Breast Cancer | RPMI, 10% FBS, Pen-Strep | ATCC CRL-2330 |
| MDA-MB- | Breast Cancer | Liebovitz's L-15, 10% FBS, Pen-Strep | ATCC HTB-132 |
| 468 | | | |
| MDA-MB- | Breast Cancer | Liebovitz's L-15, 10% FBS, Pen-Strep | ATCC HTB-131D |
| 453 | | | |
| HCC1806 | Breast Cancer | RPMI, 10% FBS, Pen-Strep | ATCC CRL-2335 |
| A2780 | Ovarian Cancer | RPMI, 10% FBS, Pen-Strep | Dr. Scott Kaufmann, |
| | | | Mayo Clinic |
| OVCAR5 | Ovarian Cancer | RPMI, 10% FBS, Pen-Strep | Dr. Scott Kaufmann, |
| | | | Mayo Clinic^{c} |
| IGROV-1 | Ovarian Cancer | McCoy's 5A, 10% FBS, Pen-Strep | Dr. Scott Kaufmann, |
| | | | Mayo Clinic |
| OVCAR8 | Ovarian Cancer | RPMI, 10% FBS, Pen-Strep | Dr. Scott Kaufmann, |
| | | | Mayo Clinic |
| T24 | Bladder Cancer | McCoy's 5A, 10% FBS, Pen-Strep | ATCC HTB-4 |
| RT4 | Bladder Cancer | McCoy's 5A, 10% FBS, Pen-Strep | ATCC HTB-2 |
| TCCSUP | Bladder Cancer | MEM in EBSS, 10% FBS, Pen-Strep, NEAA, | ATCC HTB-5 |
| | | 1 mM sodium pyruvate | |
| J28 | Bladder Cancer | MEM in EBSS, 10% FBS, Pen-Strep, NEAA, | ATCC |
| | | 1 mM sodium pyruvate | |
| SQ-20B | Head/neck | DMEM/F12, 10% FBS, Pen-Strep, 400 ng/mL | Dr. Mark Herzberg, |
| | cancer | hydrocortisone | University of |
| | | | Minnesota |
| JSQ3 | Head/neck | DMEM/F12, 10% FBS, Pen-Strep | Dr. Mark Herzberg, |
| | cancer | | University of |
| | | | Minnesota^{b} |
| TR146 | Head/neck | DMEM/F12, 10% FBS, Pen-Strep, 400 ng/mL | Dr. Mark Herzberg, |
| | cancer | hydrocortisone | University of |
| | | | Minnesota |
| SCC58 | Head/neck | DMEM/F12, 10% FBS, Pen-Strep, 400 ng/mL | Dr. Mark Herzberg, |
| | cancer | hydrocortisone | University of |
| | | | Minnesota |

^{a}Abbreviations: FBS: fetal bovine serum; Pen-Strep: 100 U/mL penicillin and streptomycin; EGF: epidermal growth factor; BPE: bovine pituitary extract; EBSS: Earl's balanced salt solution; NEAA: 1x non-essential amino acids.
^{b}Weichselbaum et al., 1988, Int. J. Radiat Oncol. Biol. Phys. 15: 575-579.
^{c}Monks et al., 1991, J. Natl. Cancer Inst. 83: 757-766.

### PMA Induction and PKC-NFκB Inhibitors

For induction experiments, 2.5×10⁵ cells were plated in a 6-well plate 1 day prior to drug treatment. PMA was then added to the media and incubated at 37° C. with 5% CO₂ for six hours unless otherwise indicated. Cells were harvested, RNA was extracted, and RT-qPCR was performed as previously reported (Refsland et al. 2010, Nucleic Acids Res 38:4274-4284). For experiments utilizing inhibitors, cells were pretreated with inhibitors 30 minutes prior to PMA induction (25 ng/mL). PMA (Thermo Fisher Scientific, Inc., Waltham, Mass.), cyclohexamide (Acros Organics, Thermo Fisher Scientific, Inc., Waltham, Mass.), Gö6983 (Cayman Chemical co., Ann Arbor, Mich.), LY294002 (EMD Chemicals, Merck KGaA, Darmstadt, Germany), U0126 (EMD Chemicals, Merck KGaA, Darmstadt, Germany), AEB071 (Medchemexpress, Monmouth Junction, N.J.), Gö6976 (Enzo Life Sciences, Inc., Farmingdale, N.Y.), BAY 11-7082 (R&D Systems, INc., Minneapolis, Minn.), and MG132 (Thermo Fisher Scientific, Inc., Waltham, Mass.) were stored as recommended. NIKi/Compound 31 was synthesized as described (Li et al., 2013, Bioorganic & Medicinal Chemistry Letters 23:1238-1244).

### Immunoblotting

The development and validation of the rabbit monoclonal antibody (mAb) against APOBEC3B is as described elsewhere (U.S. Provisional Patent Application No. 62/186,109, filed Jun. 29, 2015). The mAb used (referred to as 5210-87-13) effectively binds endogenous APOBEC3B in a variety of assays. The anti-tubulin antibody was obtained from Covance Inc., Princeton, N.J.

### Deaminase Activity Assays

Deaminase activity assays were performed as previously reported (Vieira et al., 2014, mBio 5(6):e02234-14). In short, 4 pmol of a fluorescently labeled oligo with a single target cytosine (5 -ATTATTATTATTCAAATGGATTTATTTATTTATTTATTTATTT-fluorescein, SEQ ID NO:2) was treated with cell extract containing 0.025 U/rxn UDG (New England BioLabs, Inc., Ipswich, Mass.), UDG buffer, and 1.75 U/rxn RNase A (Qiagen, Hilden, Germany) for two hours. Abasic sites were cleaved by treatment with 100 mM NaOH at 95° C. for 10 minutes. Substate was separated from product using 15% TBE-urea gel electrophoresis. Gels were scanned using a FujiFilm Image Reader FLA-7000.

### RNA Sequencing Experiments

Two sets of MCF10A cells in duplicate were treated every eight hours with media supplemented with PMA or DMSO for 48 hours. At 48 hours, RNA was extracted using an RNeasy Mini Kit (Qiagen, Hilden, Germany). Total RNA was submitted to the University of Minnesota Genomics Center for sequencing on the Illumina HiSeq 2000 platform. Raw reads were analyzed using both DESeq2 (Love et al., 2014, Genome Biol. 15:550) and the Tuxedo suite (Trapnell et al., 2012, Nat Protoc.7:562-578) to identify changes in mRNA expression in PMA treated versus untreated cells.

### Chromatin Immunoprecipitation Experiments

MCF10A cells were treated with either DMSO or 25 ng/mL PMA for two hours. Cross-linking was performed with 1% formaldehyde for 10 minutes at room temperature and quenched with 150 mM glycine. Cells were then lysed in Farnham Lysis Buffer at 4° C. for 30 minutes. Nuclei were pelleted, resuspended in RIPA Buffer, and sonicated (BIORUPTOR Pico, Diagenode S. A., Liège, Belgium) to generate approximately 600 bp DNA fragments. Immunoprecipitations were done using Protein G Dynabeads (Invitrogen, Thermo Fisher Scientific, Inc., Waltham, Mass.) and 2 μg antibody per sample. Samples were washed in 1 mL low salt wash buffer, 1 mL high salt wash buffer, 1 mL LiCl wash buffer, and eluted at 65° C. for 30 minutes. Samples were reverse cross-linked using 200 mM NaCl and treated with Proteinase K for 12 hours at 65° C. DNA was purified using a ChIP DNA Clean and Concentrator Kit (Zymo Research Corp., Irvine, Calif.) and qPCR was performed with SYBR Green master mix (Roche Diagnostics USA, Indianapolis, Ind.) on a Roche LightCycler 480. Values represent the percentage of input DNA immunoprecipitated (IP DNA) and are the average of three independent qPCR reactions.

ChIP reagents are listed in Table 2.

TABLE 2
ChIP reagents

| Category | Reagent | Description |
|---|---|---|
| Antibody | Rabbit IgG | sc-2027, Santa Cruz Biotechnology, |
| | | Inc., Dallas, TX |
| Antibody | RNA Pol II (Ser 5) | ab5131, Abcam plc, Cambridge, |
| | | United Kingdom |
| Antibody | Rel A (p65) | sc-372, Santa Cruz Biotechnology, |
| | | Inc., Dallas, TX |
| Antibody | Rel B | sc-226, Santa Cruz Biotechnology, |
| | | Inc., Dallas, TX |
| Buffer | Farnham Lysis buffer | 5 mM PIPES pH 8 |
| | | 85 mM KCl |
| | | 0.5% Nonidet P-40 |
| | | 1x EDTA-free Protease Inhibitor |
| | | Cocktail (Roche) |
| Buffer | RIPA buffer | 50 mM Tris-HCl pH 8 |
| | | 150 mM NaCl |
| | | 5 mM EDTA |
| | | 1% Nonidet P-40 |
| | | 0.5% deoxycholate |
| | | 0.1% SDS |
| | | 1x EDTA-free Protease Inhibitor |
| | | Cocktail (Roche) |
| Buffer | Low salt wash buffer | 20 mM Tris-HCl pH 8 |
| | | 150 mM NaCl |
| | | 2 mM EDTA |
| | | 0.1% SDS |
| | | 1% Triton X-100 |
| Buffer | High salt wash buffer | 20 mM Tris-HYCl pH 8 |
| | | 500 mM NaCl |
| | | 2 mM EDTA |
| | | 0.1% SDS |
| | | 1% Triton X-100 |
| Buffer | LiCl wash buffer | 20 mM Tris-HCl pH 8 |
| | | 0.5M LiCl |
| | | 1% Nonidet P-40 |
| | | 1% deoxycholate |
| | | 1 mM EDTA |
| Buffer | Elution buffer | 100 mM NaHCO₃ |
| | | 1% SDS |

### Example 2

### Animal Model

Human cancer cell lines, such as those listed above, and many others, upon engraftment into mice (e.g., subcutaneously, intraperotoneally, or otherwise), provide model systems for human tumor evolution, heterogeneity, and drug resistance. For instance, the estrogen-receptor positive breast cancer cell line MCF7L is a model for endocrine therapy and resistance.

The MCF-7 system may be adapted for evaluating APOBEC3 mutagenesis and drug resistance by creating derivative lines expressing low APOBEC3B (e.g., shRNA transduced) and high APOBEC3B (empty vector transduced) using lentivirus transduction followed by selection with 1 μg/ml puromycin for one week (Burns et al., 2013, Nature 494:366-370) and determine whether endogenous APOBEC3B contributes to the development of tamoxifen resistance. Five-million engineered cells are injected subcutaneously (xenografted) into athymic/ovariectomized nude-Foxnlnu (4-5 weeks old, The Jackson Laboratory, Bar Harbor, Me.). Each animal's drinking water is supplemented with 1 μM estradiol to provide continuous hormonal stimulation. The xenografted cells form large tumors within 150 days post-engraftment and depleting endogenous APOBEC3B causes a modest delay in tumor growth (dark square versus dark circle symbols in FIG. 8).

500 μg tamoxifen treatment 5 days/week by subcutaneous injection arrested the growth of both derivative lines for several months (light square and light circle symbols in FIG. 8). However, cells expressing high levels of APOBEC3B invariably developed resistance to tamoxifen and grew into large tumors (light squares), whereas only one of the APOBEC3B low cell masses became resistant over the year-long duration of this experiment (light circles). These data demonstrate that the DNA mutating enzyme APOBEC3B contributes to the development of tamoxifen-resistance in this xenograft model system.

The MCF-7 system can be adapted further to investigate the effect of administering a PKC-NFκB axis inhibitor on APOBEC3B expression. In addition to the treatments described above (i.e., with or without tamoxifen), the mice are treated with administering a PKC-NFκB axis inhibitor, resulting in decreased tumor volume compared to A3B^{low} and A3B^{low}+TAM in FIG. 8, respectively.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of treating a subject having or at risk of having a tumor, the method comprising:
administering to the subject an amount of a PKC-NFκB axis inhibitor effective to ameliorate at least one symptom or clinical sign of the tumor.

2. The method of claim 1 wherein the PKC-NFκB axis inhibitor comprises a PKC inhibitor.

3. The method of claim 3 wherein the PKC inhibitor comprises Gö6983, Gö6976, MT477, RO 32-0432, myr-FARKGALRQ, chelerythrine, RO 31-7549, safingol, Compound 3, Compound 8, aprinocarsen, balmoralmycin (I), bisindolylmaleimides, or sotrastaurin.

4. The method of claim 1 wherein the PKC-NFκB axis inhibitor comprises an NFκB inhibitor.

5. The method of claim 4 wherein the NFκB inhibitor comprises TPCA-1.

6. The method of claim 1 wherein the PKC-NFκB axis inhibitor comprises a proteasome inhibitor.

7. The method of claim 6 wherein the proteasome inhibitor comprises BAY 11-7082, MG132, bortezomib, salinosporamide A, or carfilzomib.

8. The method of claim 1 wherein the PKC-NFκB axis inhibitor comprises a NIK inhibitor.

9. The method of claim 1, wherein the tumor comprises a tumor resulting from acute lymphoblastic leukemia (ALL), bladder cancer, breast cancer, cervical cancer, chondrosarcoma, chronic lymphocytic leukemia (CLL), esophageal cancer, head and neck cancer, kidney cancer, lung cancer, B cell lymphoma, melanoma, myeloma, osteosarcoma, ovarian cancer, pancreatic cancer, stomach cancer, thyroid cancer, uterine cancer, or uveal cancer.

10. A method of treating a subject having a tumor, the method comprising:
confirming that APOBEC3B is present in cells of the tumor; and
administering to the subject an amount of a PKC-NFκB axis inhibitor effective to decrease APOBEC3B in the cells of the tumor.

11. The method of claim 10 wherein the presence of APOBEC3B in the cells of the tumor is assayed by RT-qPCR, detecting an APOBEC3B mutation signature through DNA sequencing, or detecting the protein itself using an APOBEC3B-specific antibody.

12. The method of claim 10 wherein the PKC-NFκB axis inhibitor is administered to the subject after the subject receives another anti-tumor therapy.

13. The method of claim 10 wherein the PKC-NFκB axis inhibitor is administered to the subject before the subject receives another anti-tumor therapy.

14. The method of claim 10 wherein the PKC-NFκB axis inhibitor is administered to the subject concurrent with the subject receiving another anti-tumor therapy.

15. The method of claim 12, wherein the tumor therapy comprises chemotherapy, targeted therapy, immunotherapy, radiotherapy, or palliative care.

16. The method of claim 13, wherein the tumor therapy comprises chemotherapy, targeted therapy, immunotherapy, radiotherapy, or palliative care.

17. The method of claim 14, wherein the tumor therapy comprises chemotherapy, targeted therapy, immunotherapy, radiotherapy, or palliative care.


SEQUENCE LISTING (5 sequences)

SEQ ID NO: 1 - protein, 10 aa, Homo Sapiens
GGRRNNYYCC

SEQ ID NO: 2 - DNA, 43 bp, Artificial
  Polynucleotide
attattattattcaaatggatttatttatttatttatttattt

SEQ ID NO: 3 - protein, 26 aa, Artificial
  Polynucleotide
AAVALLPAVLLALLAPVQRKRRKALP

SEQ ID NO: 4 - protein, 9 aa, Artificial
  Polypeptide
  MISC_FEATURE: 2,3-Diaminopropionic Acid
  misc_feature (8)..(8): Xaa can be any naturally occurring amino acid
ARRGALRXA

SEQ ID NO: 5 - protein, 9 aa, Artificial
  Polypeptide
  MISC_FEATURE: 2,3-Diaminopropionic Acid
  misc_feature (8)..(8): Xaa can be any naturally occurring amino acid
ARRGALRXA